(19) United States
(12) Patent Application Publication
(10) Pub. No.: US 2025/0255735 A1
(43) Pub. Date: Aug. 14, 2025

(54) HIGH-PERFORMANCE PROSTHETIC ATTACHMENT

(71) Applicant: Philip Smith, Valrico, FL (US)

(72) Inventor: Philip Smith, Valrico, FL (US)

(21) Appl. No.: 18/438,929

(22) Filed: Feb. 12, 2024

(51) Int. Cl. A61F 2/66; A61F 2/50; A61F 2/68; A61F 2/70

(52) U.S. Cl. CPC A61F 2/6607; A61F 2002/5038; A61F 2002/5083; A61F 2002/6614; A61F 2002/6863; A61F 2002/704

(57) ABSTRACT

Aspects relate to a high-performance prosthetic attachment, the prosthetic attachment may include an articulating attachment with varying, adjustable ranges of motion, a limb connector coupled to the articulating attachment, a base connector coupled to the articulating attachment, and a base. Further embodiments of the high-performance prosthetic attachment may include at least an actuator mechanically connected to the articulating attachment and configured to adjust the range of motion of the articulating attachment, and at least a sensor, configured to detect attachment datum of a base. Lastly, further embodiments may include a control circuit, which may be communicatively connected to the prosthetic attachment and configured to receive attachment data and drive the actuator to adjust the range of motion available to the articulating attachment.

### FIELD OF THE INVENTION

The present invention generally relates to the field of high-performance prosthetics. In particular, the present invention is directed to a high-performance prosthetic attachment.

### BACKGROUND

Use of high-performance prosthetics, or sports prosthetics for athletes, has grown increasingly popular in use as demand for refined and targeted prosthetics grows. The demand has been met with many technological advancements, allowing athletes requiring assistance from prosthetics to not just participate in their chosen sport, but excel in them. However, high-performance prosthetics generally are quite expensive and usually fairly niche in their application.

### SUMMARY OF THE DISCLOSURE

In an aspect, a high-performance prosthetic attachment, may include an articulating attachment with varying, adjustable ranges of motion, a limb connector coupled to an articulating attachment, a base connector coupled to the articulating attachment, and a base. Further embodiments of the high-performance prosthetic attachment may include at least an actuator mechanically connected to the articulating attachment and configured to adjust the range of motion of the articulating attachment, and at least a sensor, configured to detect attachment datum of a base. Lastly, further embodiments may include a control circuit, which may be communicatively connected to the prosthetic attachment and configured to receive attachment data and drive the actuator to adjust the range of motion available to the articulating attachment.

In another aspect, a high-performance prosthetic attachment may further include a base with at least a sensor located thereon. The base itself may be permanently attached to base connector and/or the base may be removeable. Additionally, the base may be bolted to a plate which may be bolted to the base connector and coupled with the prosthetic attachment.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1A is an exemplary embodiment of a high-performance prosthetic attachment;

FIG. 1B is a top-down overview of an exemplary embodiment of a high-performance prosthetic attachment;

FIG. 1C is an exemplary embodiment of a high-performance prosthetic attachment with an inline-skate-type base;

FIG. 1D is an exemplary embodiment of a high-performance prosthetic attachment with a ice-skate-type base;

FIG. 2 is a block diagram illustrating an exemplary embodiment of a high-performance prosthetic attachment;

FIG. 3 is an exemplary machine-learning module;

FIG. 4 is an exemplary neural network;

FIG. 5 is an exemplary node of a neural network; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems for high-performance prosthetic attachments. In an embodiment, a high-performance prosthetic attachment is configured to allow prosthetic users the ability to participate in a given sport with the assistance of a prosthetic. Participation need not be at a competitive level but may rather be for leisure or fun. Specifically, in an embodiment, a high-performance prosthetic attachment may allow a prosthetic user the ability to participate in a variety of sports and/or activities. This may be accomplished through the use of a high-performance prosthetic attachment due to its variability in range of motion in detection of a specific base and its corresponding ranges of motion. As used in this disclosure, “prosthetic” is a man-made substitute for a missing body part. Likewise, as used in this disclosure, a “prosthetic user” is a person who is missing a body part and requires the assistance of a prosthetic.

Aspects of the present disclosure can be used to increase prosthetic users' access and ability to participate in any given sport. Aspects of the present disclosure can also be used to increase the comfort and safety of a prosthetic users' participation in any given sport. This is so, at least in part, because the control circuit, which is communicatively connected to the prosthetic attachment, is able to detect attachment data and determine the range of motion necessary for participation in a given sport by relation to the base that is attached. This may allow for an attachment that may be used with various prosthetic bases, whether sports related or for everyday use.

In the following description, for the purpose of explanation, numerous specific details are set forth in order to provide a thorough understanding of the present invention. Exemplary embodiments illustrating aspects of the present disclosure are described below in the context of several specific examples. Furthermore, there is no intention to be bound by any expressed or implied theory presented in this disclosure.

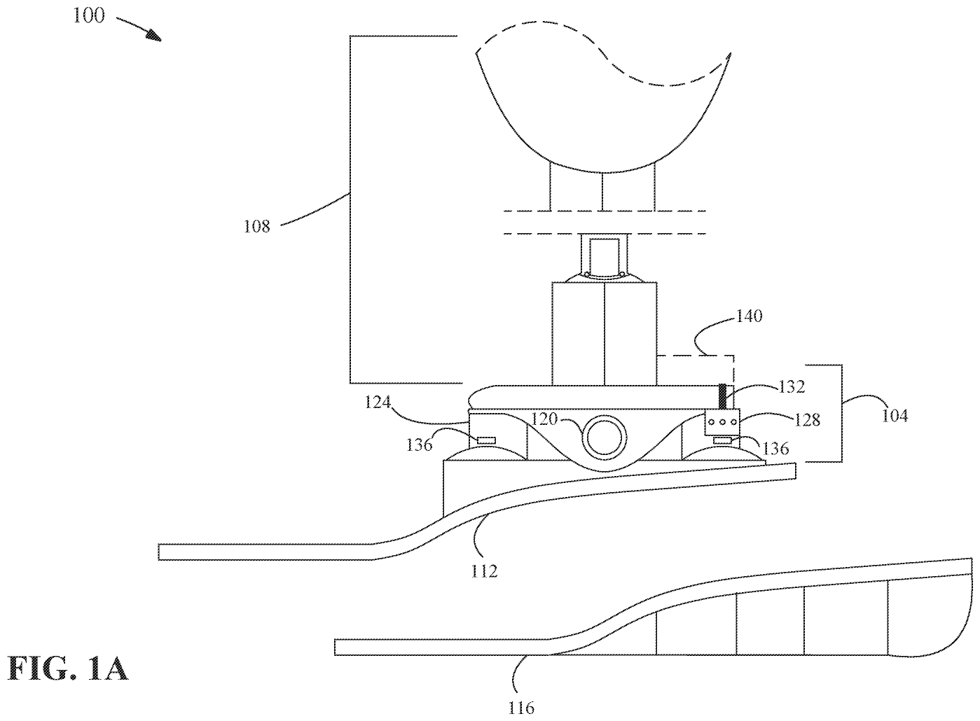

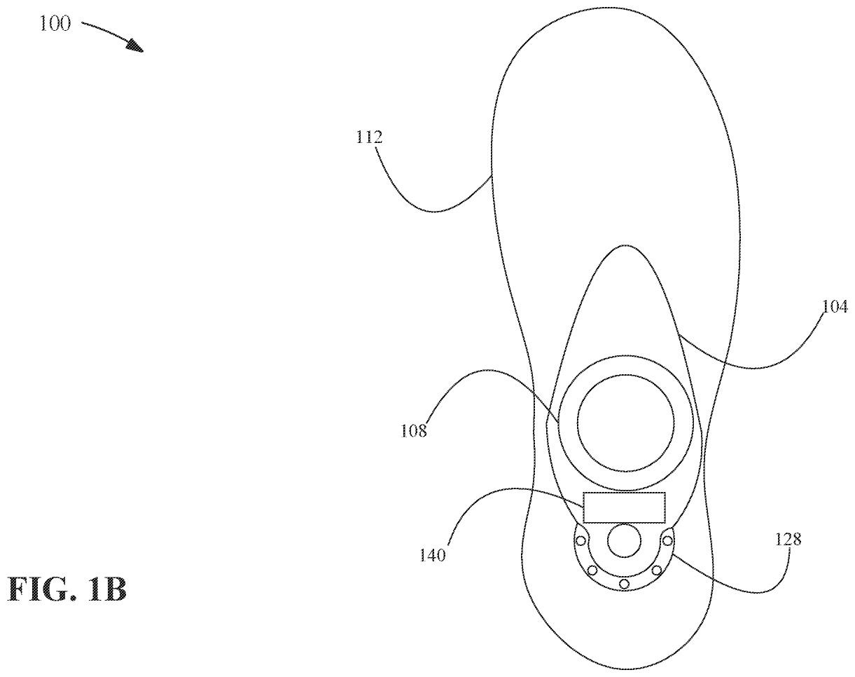

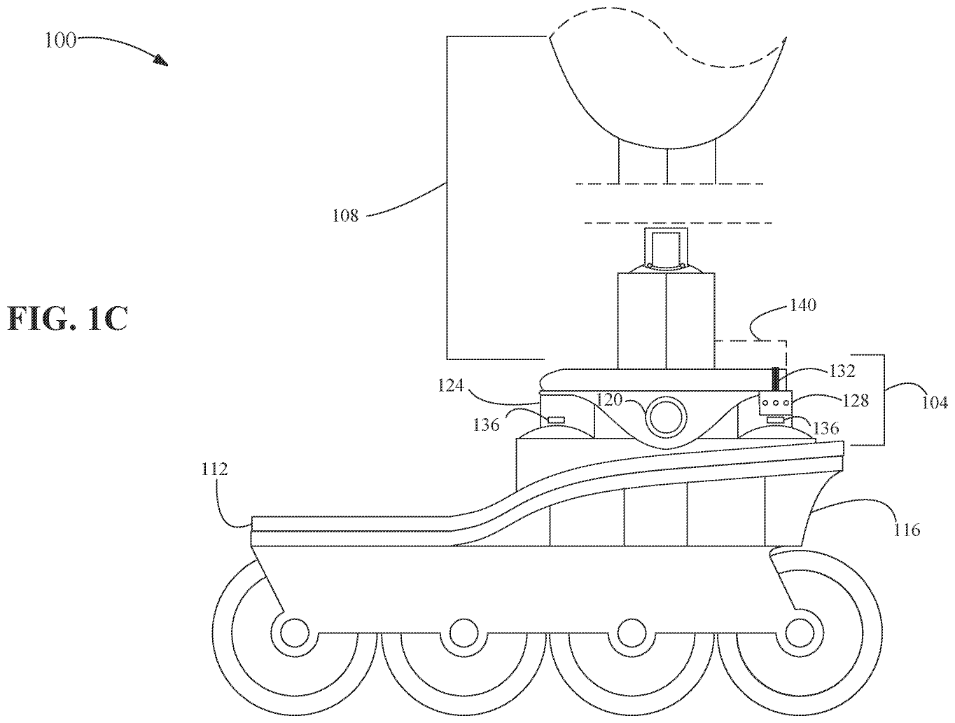

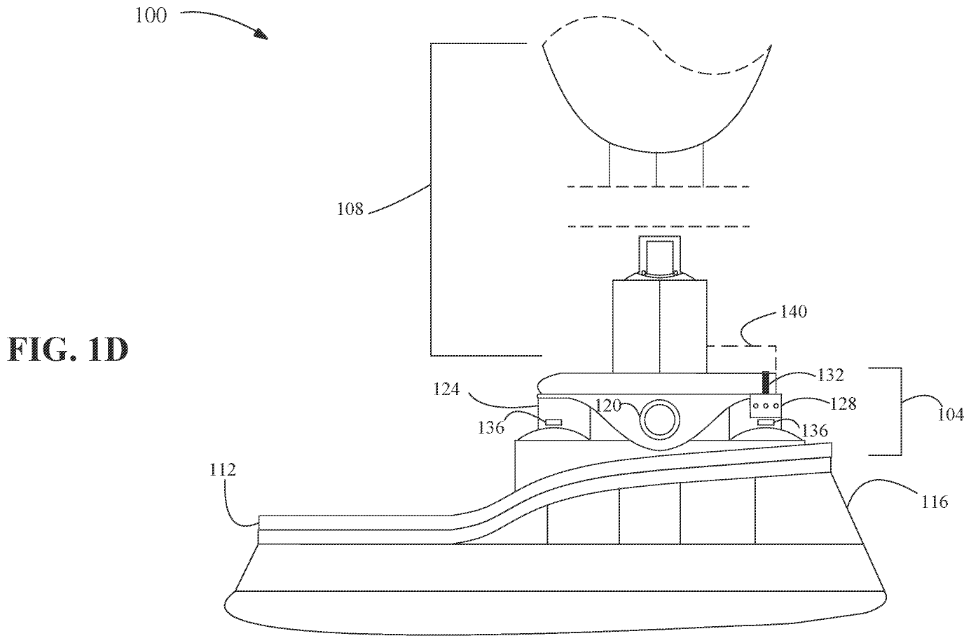

Referring now to FIG. 1A, an exemplary embodiment of a high-performance prosthetic attachment 100 is illustrated. In an embodiment, high-performance prosthetic attachment 100 may include an articulating attachment 104. Articulated attachment 104 may have varying, adjustable ranges of motion. High-performance prosthetic attachment 100 may include limb connector 108 coupled to articulating attachment 104, base connector 112 coupled to articulating attachment 104, and base 116. Further embodiments of high-performance prosthetic attachment 100 may include at least an actuator 132 mechanically connected to articulating attachment 104 and configured to adjust a range of motion of the articulating attachment 104, and at least a sensor 136, configured to detect attachment datum of base 116. Lastly, further embodiments may include a control circuit 140, which may be communicatively connected to high-performance prosthetic attachment 100 and configured to receive attachment data and drive actuator 132 to adjust a range of motion available to articulating attachment 104.

Still referring to FIG. 1A, at least an articulating attachment 104 may include a multi-axial joint and/or a hinged synovial joint. As used in this disclosure, a “multi-axial joint” is a joint that allows for three types of movement: anterior-posterior, medial-lateral, and rotational. In an embodiment a multi-axial joint may include rotary multi-axial joints, rolling-hinge joints, spherical joints, polycentric joints, and the like. In some embodiments, a multi-axial joint may be made of metal material, such as steel, carbon fiber, polymers, hydraulic systems, and/or the like. A “hinged synovial joint,” as used in this disclosure, is a joint that exists in the body, such as the ankle and/or wrist, and serves to allow motion primarily in one plane. Likewise, “synovial joints” allow for smooth movement between adjacent materials, such as other bones in the body. In an embodiment, at least an articulating attachment 104 may include a hinged synovial joint embodied in metal, such as steel, carbon fiber, polymers, hydraulic systems, and/or the like.

Still referring to FIG. 1A, in some cases, at least an articulating attachment 104 may also include a hydraulic joint. A “hydraulic joint,” as described herein, is a joint that employs a fluid-based system to facilitate movement. In one or more embodiments, hydraulic fluid within hydraulic joint may be pressurized to different levels, allowing for adjustable resistance and range of motion. In a non-limiting example, hydraulic joint may be particularly beneficial in adapting to more difficult activities and complex terrains compared to other types of joint as described herein. In some cases, hydraulic joint may be constructed using materials such as high-grade aluminum, stainless steel, or advanced polymers, which provide both strength and light weight properties. In some cases, hydraulic joint may also include one or more adjustable valves for controlling fluid flow; for instance, and without limitation, user may be able to customize the joint's stiffness and damping characteristics according to individual preferences and needs by manipulating the adjustable valves through an externally accessible adjustment knob or screw (e.g., truing the adjustment knob clockwise the flow of hydraulic fluid may be restricted, increasing the resistance of the joint). In other cases, an electronic control system (e.g., control circuit as described below in further detail below) may be incorporated herein for valve adjustment.

With continued reference to FIG. 1A, additionally, or alternatively, at least an articulating attachment 104 may incorporate a microprocessor ankle unit. As used in this disclosure, a “microprocessor ankle unit” is a component that employs one or more sensors and microprocessors to dynamically adjust ankle's position and resistance in real-time. In some cases, microprocessor ankle unit may allow for a more natural gait pattern thereby improving the user's stability and comfort, especially on uneven surface or during varying movement speeds. Exemplary sensors such as, without limitation, accelerometers, gyroscopes, and/or the like May be used to accurately detect user's movement and terrain changes. In a non-limiting example, one or more microprocessor may make instantaneous adjustments to ankle's angle and resistance based on received sensor data. In some cases, microprocessor ankle unit may be constructed from lightweight materials such as titanium or carbon fiber. In one or more embodiments, microprocessor ankle unit may automatically adjust prosthetic's response based on data related to user's activity level, for example, and without limitation, when walking or running, user may adjust the prosthetic to different stiffness levels to accommodate the impact levels associated with user's walking or running speed (e.g., a softer setting may be used for walking while a firmer setting may be selected for running).

Furthermore, and further referring to FIG. 1A, the range of movement provided by articulating attachment 104 may be adjustable. “Articulating,” as used in this disclosure, is to form a joint. An exemplary embodiment of this may include an articulating attachment 104 that joins with base connector 112 to form a prosthetic joint imitating the ankle joint. In some embodiments articulating attachment 104 may include rubber bumpers, which may be configured to restrict a range of movement thereof to a specific range necessary to provide stability and yet flexibility similar to a biological human ankle. Alternatively, in some embodiments articulating attachment 104 may include hydraulics and/or a hydraulic system, configured to do the same. A specific range of movement necessary to provide stability and yet flexibility may be different for varying bases 116. For example, range of movement may be larger in a ski-type base 116, compared to an ice-skate-type base 116. Additionally, range of movement may differ for specific motions of an ankle including, without limitation, dorsiflexion, plantarflexion, inversion, eversion, and medial and lateral rotation. As used in this disclosure, “dorsiflexion” is flexion in the dorsal direction, for example the flexion of a foot towards the shin. Alternatively, “plantarflexion,” as used in this disclosure, refers to flexion of the foot away from the shin, for example by pointing one's toes down. Typically, generally accepted ranges of motion for the ankle may include plantar flexion of 40 degrees, and dorsiflexion of 20 degrees. As used in this disclosure, “inversion” is the movement of the ankle so that the plantar area points medially. For example, when one rolls their ankle laterally, or to correct balance when walking on uneven terrain facing the sole of the foot towards the midline of the body. Alternatively, “eversion” as used in this disclosure, is the movement in which the foot rotates so that the sole faces away from the midline of the body. Generally accepted ranges of inversion may include 30 degrees, and eversion of 20 degrees. “Medial and lateral rotation” as used in this disclosure, relate to the rotation of body parts towards or away from the center of the body. “Medial rotation” is an internal rotation, whereas “lateral rotation” is an external rotation. Range of rotation on a vertical access may include anywhere from 45 degrees to 90 degrees.

With continued reference to FIG. 1A, at least an articulating attachment 104 may include a center axis of articulation, embodying joint-like capabilities, as well a resistance system acting as a form of resistance to plantar flexion and dorsiflexion. As used in this disclosure, “center axis of articulation” is the point at which rotation and range of motion are accomplished within articulating attachment 104. The center axis of articulation may be inline or slightly forward to the prosthetic user's weight line. Joint-like capabilities may include various types of movement, including anterior-posterior, medial-lateral, and/or rotational movements. Additionally, joint-like capabilities may include the ability to provide both flexibility and stability for a given activity. In an embodiment, a resistance system may include the use of polymer bushings. Additionally, in an embodiment, polymer bushings may utilize a passive pneumatic hydraulic system to maintain stability of articulating attachment 104. In an embodiment a resistance system of the articulating attachment 104 may otherwise utilize hydraulic resistance.

With further reference to FIG. 1A, cutouts of material, and/or voids, may be made to provide a light-weight prosthetic to prosthetic users. For example, and without limitation, a cutout of articulating attachment 104 may be made above the center axis of articulation in a way that does not compromise the stability of the unit configured to bear the weight of a prosthetic user. Additionally, where necessary material may be added for the safety of the user. For example, a lip may be added to the front of articulating attachment 104 in order to safeguard the user from catching their fingers in the moving parts of the articulating attachment 104.

Continuing to reference FIG. 1A, at least an articulating attachment 104 may include an adjustment wheel. An adjustment wheel of articulating attachment 104 may be located at the rear of articulating attachment 104 and configured to adjust range of movement required to maintain stability and necessary flexibility for any given base 116. Adjustment of the wheel may occur manually or through the use of actuator 132 as described later in this disclosure.

Continuing to reference FIG. 1A, in an embodiment, limb connector 108 may include any embodiment of limb connector 108 as described within this disclosure. “Limb connector,” as used in this disclosure, is the interface between the limb remnant and the prosthetic. The portion of prosthetic attachment that fits snugly over the limb remnant may be a part of limb connector 108, and is referred to as the “socket,” and determines the prosthetic user's comfort and ability to control the prosthetic. Exemplary embodiments of limb connector 108 may include various embodiments of a socket such as, without limitation, transtibial sockets, including sockets that are patellar tendon bearing and/or total surface bearing, and transfemoral sockets, including sockets that are quadrilateral and/or ischial containment. In some embodiments, limb connector 108 may include a socket liner made of silicone elastomers. Additionally, in some embodiments socket liners may be tethered to the inside of a socket with a mechanical device called a shuttle lock; a shuttle lock may provide suspension for a prosthetic limb. As used in this disclosure, “suspension” refers to the process of maintaining an adequate connection between limb connector 108 and a limb remnant, configured to ensure a prosthetic does not fall off during use. In some embodiments, limb connector 108 may include the aforementioned socket liner paired with a suspension embodiment known as a lanyard. As used here, “lanyard” is when a cord or strap is attached to the lower end of the liner to provide suspension for a prosthetic. Additional embodiments of limb connector 108 may include various forms of suspension, such as without limitation, cuffs, straps, and belts, self-suspending sockets, external sleeves, pin and lock suspensions, suction with or without a liner, vacuum-assisted suspension, and or osseointegration. “Osseointegration” as used in this disclosure, is a procedure that places an implant in the bone, allowing for direct attachment to a prosthetic without the need for a socket or a liner. Limb connector 108 may include any one or any combination of the disclosed embodiments of sockets, liners, and/or lack thereof.

In further reference to FIG. 1A, limb connector 108 may additionally include a portion of prosthetic attached to a socket and further configured to attach to articulating attachment 104. This portion of limb connector 108 may be referred to as the “modular transtibial system.” In an embodiment, a modular transtibial system may include one or more prosthetic pyramid adapter sets with male and female counterparts, a pylon, one or more pylon adapters, and one or more clamp adapters. As used in this disclosure, a “prosthetic pyramid adapter set” or “pyramid adapter set” is a receptacle for coupling a prosthetic limb to a socket, including a unitary machined component formed with an externally threaded neck section flanged by a larger-diameter disk. A “pylon,” as used in this disclosure, is a rigid, usually tubular structure between a socket and/or knee unit and a foot that provides a weight bearing shock-absorbing support shaft for a prosthetic. Both “pylon adapters,” and “clamp adapters” as used in this disclosure refer to parts of a prosthetic that assist in the stable connection between a socket and the rest of a prosthetic, such as articulating attachment 104.

Still referring to FIG. 1A, limb connector 108 may be coupled to at least an articulating attachment 104. As used in this disclosure, “coupling” is the act of bringing or coming together. In some embodiments, coupling may be mechanical, including configurations that include without limitation, latching mechanisms, threaded adapters, and/or adhesive and bonding mechanisms. In other embodiments coupling may be configured by a magnetic mean. Additionally, in some embodiments, coupling may be accomplished by any one or combination of the embodiments previously disclosed. For example, in an embodiment, coupling of limb connector 108 and at least articulating attachment 104 may be accomplished with a pyramid adapter set. In this embodiment a male part of the pyramid adapter set may be located on articulating attachment 104, whereas a female part of a pyramid adapter set may be located on the distal end of limb connector 108.

With continued reference to FIG. 1A, high-performance prosthetic attachment 100 may include base connector 112. In an embodiment base connector 112 may include any embodiment of base connector 112 as described within this disclosure. Base connector 112 in some embodiments may be made of lightweight material capable of supporting a high threshold of weight. For example, base connector 112 may be made of carbon fiber and/or a similarly durable lightweight material. Furthermore, base connector 112 may be configured in a way that imitates a footplate that is contoured to match a specific and/or general base 116. In an embodiment, base connector 112 may be textured and/or a smooth surface. Additionally, base connector 112 may be coupled to at least an articulating attachment 104. In some embodiments, coupling may be mechanical, including configurations that include without limitation, latching mechanisms, threaded adapters, and/or adhesive and bonding mechanisms. In other embodiments coupling may be configured by a magnetic attachment. Additionally, in some embodiments, coupling may be accomplished by any one or combination of the embodiments previously disclosed. Base connector 112 may be contoured to match a specific base 116 configured to accomplish a specific sport and/or task. For example, base connector 112 may be configured to match a standard hockey skate and figure skate chassis. “Chassis,” as used in this disclosure is the base structural framework of a given structure.

Still referring to FIG. 1A, in some embodiments, base connector 112 may be configured to directly couple with base 116 or a prosthetic piece. Alternatively, in other embodiments, base connector 112 may be coupled to a plate that may be configured to couple with base 116 or a prosthetic piece. “Prosthetic piece” and “base,” as used in this disclosure is any attachment to base connector 112, configured to aid a prosthetic user in participating in a given task. Even further, in some embodiments, base connector 112 may be configured to couple with a plate attached to base 116 or a prosthetic piece. A plate in any of these embodiments may additionally be embodied in various materials. For instance, a plate may be made of metal. Alternatively, the plate may be made of carbon fiber. In some embodiments, coupling may be mechanical, including configurations that include without limitation, latching mechanisms, threaded adapters, and/or adhesive and bonding mechanisms. Additional attachments and/or coupling mechanisms may include, but are not limited to, snap-fit or snap-on connectors, slide-and-lock mechanisms, clamps, straps, and the like. In other embodiments coupling may be configured by magnetic attachments. Additionally, in some embodiments, coupling may be accomplished by any one or combination of the embodiments previously disclosed.

Referring still to FIG. 1A, in some embodiments, base connector 112 may be communicatively connected to articulating attachment 104. As used in this disclosure, “communicatively connected” means connected by way of a connection, attachment or linkage between two or more relata which allows for reception and/or transmittance of information therebetween. For example, and without limitation, a connection may be wired or wireless, direct or indirect, and/or between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Continuing to reference FIG. 1A, high-performance prosthetic attachment 100 may further include base 116. Base 116 may include any prosthetic piece and may be coupled in any manner as previously disclosed in this disclosure. Additionally, in some embodiments base connector 112 may be part of a prosthetic piece, configured in a way in that it is permanently connected to a prosthetic piece. In some embodiments, base 116 may include prosthetic attachments that relate to a specific task, such as without limitation a prosthetic inline skate, ice-skate, snowboard, cross-country ski, downhill ski, and/or the like. Additionally, base 116 may be communicatively connected to articulating attachment 104, configured to transmit attachment data relating to base 116. As used in this disclosure, “attachment data” refers to data relating to the type of base 116 or prosthetic piece attached to base connector 112. Attachment data may include information regarding the operating range of movement that provides the appropriate and best range to participate in the associated task. Attachment data may additionally include stability data. “Stability data” as used in this disclosure may include information relating to postural sway and/or postural balance.

Still referring to FIG. 1A, in some embodiments, high-performance prosthetic attachment 100 may further include at least an actuator 132. At least an actuator 132 may include one or more embodiments of an actuator. In an embodiment, at least an actuator 132 may include a component of a machine that is responsible for moving and/or controlling a mechanism or system. Actuator 132 may, in some embodiments, require a control signal and/or a source of energy or power. In some embodiments, a control signal may be relatively low energy. Exemplary control signal forms may include electric potential or current, pneumatic pressure or flow, or hydraulic fluid pressure or flow, mechanical force/torque or velocity, or even human power. In some embodiments, actuator 132 may have an energy or power source other than a control signal. This may include a main energy source, which may include for example electric power, hydraulic power, pneumatic power, mechanical power, and the like. In some embodiments, upon receiving a control signal, actuator 132 may respond by converting source power into mechanical motion. Furthermore, in some embodiments, actuator 132 may be understood as a form of automation or automatic control.

With continued reference to FIG. 1A, in some embodiments, actuator 132 may include a hydraulic actuator. A hydraulic actuator may consist of a cylinder or fluid motor that uses hydraulic power to facilitate mechanical operation. Output of hydraulic actuator may include mechanical motion, such as without limitation linear, rotatory, or oscillatory motion. In some cases, hydraulic actuator may employ a liquid hydraulic fluid. As liquids, in some cases. are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

With continued reference to FIG. 1A, in some embodiments, actuator 132 may include a pneumatic actuator. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

With continued reference to FIG. 1A, in some cases, actuator 132 may include an electric actuator. Electric actuator may include any of electromechanical actuators, linear motors, and the like. In some cases, actuator 132 may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. electric actuator may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least three different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

With continued reference to FIG. 1A, in some embodiments, actuator 132 may include a mechanical actuator. In some cases, a mechanical actuator may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanism, including for example without limitation gears, rails, pulleys, cables, linkages, and the like.

Still referencing FIG. 1A, at least an actuator 132 may be mechanically connected to articulating attachment 104 and configured to adjust range of movement available to articulating attachment 104. Likewise, at least an actuator 132 may be communicatively connected to control circuit 140 and configured to implement commands or signals transmitted to it by control circuit 140 and inversely from actuator 132 to control circuit 140. As used in this disclosure, a “signal” is any intelligible representation of data, for example from one device to another. A signal may include an optical signal, a hydraulic signal, a pneumatic signal, a mechanical signal, an electric signal, a digital signal, an analog signal, and the like. In some cases, a signal may be used to communicate with a computing device, for example by way of one or more ports. In some cases, a signal may be transmitted and/or received by a computing device for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With further reference to FIG. 1A, in some embodiments articulating attachment 104 may include a sensor configured to detect attachment datum. Furthermore, this embodiment may include a communicative connection to control circuit 140 which may be configured to receive an attachment datum and drive at least an actuator 132 to adjust a range of motion available to articulating attachment 104. The communicative connection may be through mechanical means and or through sensed signals and commands. As used in this disclosure, a “mechanical communicative connection” is a mechanical connection that is responsive to specific variations in the mechanical configuration of the system. For example, in an embodiment wherein base connector 112 is bolted either to a plate further bolted to articulating attachment 104 and/or directly to articulating attachment 104, the mechanical configuration creates a restriction on the range of motion for that specific configuration. Control circuit 140 may sense a mechanical communicative connection through placement of one or more sensors 136 on base 116 and base connector 112. Sensed attachment data may be communicated through a mechanical configuration through placement of one or more sensors 136 located on articulating attachment 100. Alternatively, in some embodiments the communicative connection may be accomplished through sensed attachment datum via a sensor located at any location as described within this disclosure. In some embodiments, base 116 may include at least a sensor 136 communicatively connected to control circuit 140 configured to relay information of the type of base 116 attached to base connector 112. In an embodiment, base 116 may further include a control circuit separate from control circuit 140. Communication of attachment data of base 116 and control circuit 140 may be accomplished through a variety of means, such as wireless or wired connections including, but not limited to, radio frequency identification (RFID), near-field communication (NFC), and/or the like. In some embodiments, sensor 136 located on base connector may be configured to scan an optical, RFID, and/or an NFC tag to identify the attached base 116. Further, in some embodiments, base connector 112 may be configured to sense a characteristic of base 116 that indicates its identity and/or purpose. Sensing of a characteristic of base 116 may be accomplished through use of tabs, switch circuits, button switches, and/or the like.

With continued reference to FIG. 1A, in an embodiment, at least a sensor 136 may be one and/or more embodiments of a sensor as described within this disclosure. As used in this disclosure, a “sensor” is a device that is configured to detect an input and/or a phenomenon and transmit information related to the detection. For example, and without limitation, a sensor may transduce a detected connection phenomenon and/or characteristic into a sensed signal with respect to a reference. At least a sensor 136 may detect a plurality of data. A plurality of data detected by at least a sensor 136 may include, but is not limited to, attachment data, postural sway data, postural balance data, and the like. As used in this disclosure, “attachment data” refers to any data that correlates to a determination of the necessary range of movement for any specific base 116 connection. “Postural sway data” and “postural balance data,” refer to any data that senses the system of high-performance attachment's 100 balance while in use. In one or more embodiments, and without limitation, at least a sensor 136 may include individual sensors and/or a plurality of sensors (e.g., a sensor suit or a sensor array). In one or more embodiments, and without limitation, sensor 136 may include proximity sensors, ultrasonic sensors, hall effect sensors, pressure sensors, IR sensors, load cell, accelerometers, gyroscopes, magnetometers, and the like. At least a sensor 136 may be a contact or a non-contact sensor. Signals may include electrical, electromagnetic, visual, audio, radio waves, optical, hydraulic, pneumatic, mechanical, digital, analog, or another undisclosed signal type alone or in combination. The location of at least a sensor 136 may be at any one or multiple locations as described within this disclosure.

With continued reference to FIG. 1A, at least a sensor 136 may include a plurality of independent sensors, where any number of the described sensors may be used to detect any number of physical or electrical quantities associated with communication of base 116 connection. Independent sensors may include separate sensors measuring physical or electrical quantities that may be powered by and/or in communication with another device, such as without limitation control circuit 140, articulating attachment 104, and/or actuator 132. In some embodiments, independent sensors may be used to create or as an element of training data. Sensor output and/or data derived therefrom may be displayed, without limitation in a graphical user interface, a command-line interface, or the like. In an embodiment, use of a plurality of independent sensors may result in redundancy configured to employ more than one sensor that measures the same phenomenon, those sensors being of the same type, a combination of, or another type of sensor not disclosed, so that in the event one sensor fails, the ability of sensor 136 to detect phenomenon may be maintained.

With continued reference to FIG. 1A, at least a sensor 136 may include a sensor suite which may include a plurality of sensors that may detect similar or unique phenomena. At least a sensor 136 may include a plurality of sensors in the form of individual sensors or a sensor suite working in tandem or individually. A sensor suite may include a plurality of independent sensors, as described in this disclosure, where any number of the described sensors may be used to detect any number of physical or electrical quantities associated with base connector 112 connection and/or base 116. Independent sensors may include separate sensors measuring physical or electrical quantities that may be powered by and/or in communication with circuits independently, where each may signal sensor output to a control circuit. In an embodiment, use of a plurality of independent sensors may result in redundancy configured to employ more than one sensor that measures the same phenomenon, those sensors being of the same type, a combination of, or another type of sensor not disclosed, so that in the event one sensor fails, the ability to detect phenomenon is maintained.

With continued reference to FIG. 1A, at least a sensor 136 may include a sense board. A sense board may have at least a portion of a circuit board that includes one or more sensors configured to measure or detect a sensor input. In one or more embodiments, a sense board may include one or more circuits and/or circuit elements, including, for example, a printed circuit board component. A sense board may include, without limitation, a control circuit configured to perform and/or direct any actions performed by the sense board and/or any other component and/or element described in this disclosure. The control circuit may include any analog or digital control circuit, including without limitation a combinational and/or synchronous logic circuit, a processor, microprocessor, microcontroller, or the like.

With continued reference to FIG. 1A, at least a sensor 136 is configured to transmit a sensor output signal representative of sensed information. As used in this disclosure, a “sensor signal” is a representation of a sensed information that sensor may generate. A sensor signal may include any signal form described in this disclosure, for example and without limitation, digital, analog, optical, electrical, fluidic, and the like. In some cases, a sensor, a circuit, and/or a controller may perform one or more signal processing steps on a signal. For instance, sensor, circuit, and/or controller may analyze, modify, and/or synthesize a signal in order to improve the signal, for instance by improving transmission, storage efficiency, or signal to noise ratio.

With continued reference to FIG. 1A, system, sensors, sense board, and/or a control circuit 140 as described in further detail below, may perform one or more signal processing steps on sensor signal. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

Still referring to FIG. 1A, exemplary, non-limiting methods of signal processing may include sensor input signal, signal conversion, signal representation, signal manipulation, signal analysis, signal synthesis, and signal output. As used in this disclosure, “sensor signal input” is an input signal acquired from any one or multiple sensors as disclosed in this disclosure. For example, and without limitation, a sensor signal may be acquired from any one or combination of sensor embodiment as described in this disclosure. In some embodiments sensor signal input may need to be converted to another format. For example, if the input signal is in an analog format, it may be converted into a digital format using an analog-to-digital converter (ADC). The input signal is then represented in a suitable form for processing, in some embodiments this may include representing the signal as a discrete sequence of samples or as a continuous function. In some embodiments, signal manipulation may occur, such as, without limitation, filtering, noise reduction, compression, and/or feature extraction. The manipulated signal is then analyzed to extract useful information or to make decisions. For example, without limitation, the analysis may involve detecting patterns or features in the signal, classifying the signal into different categories, and/or estimating the values of certain parameters. The output of the signal processing system may be a synthesized signal, which is generated based on the processed input signal and any additional information or constraints. An output may involve converting the signal back to an analog format using a digital-to-analog converter (DAC) or displaying the signal on a screen or speaker.

Further referring to FIG. 1A, in an embodiment, one or more components of a signal processing system may receive a signal from one or more sensors and filter the received signal to equalize and/or remove noise. Noise may be introduced at a sensor including, without limitation, by conditions under which sensing occurs, such as vibrations affecting motion sensors, distance sensor, or the like, temperature variations affecting optical sensors, electromagnetic interference affecting sensor detection of one or more phenomena to be detected, and/or shot noise. Noise may be introduced in a channel and/or communicative path from sensor to control circuit and/or other components of system. Noise may be filtered using any of the above-described signal processing techniques including without limitation adaptive filtering using Weiner, least-squares, Kalman or other filtering; notch filtering to remove, e.g., engine vibrations, and/or other processes to isolate a signal from one or more sensors. A filtered signal may be used in subsequent steps and/or processes described in this disclosure.

With further reference to FIG. 1A, in an embodiment, one or more components of a signal processing system may be improved and/or fine-tuned by using aggregations, instantiating a machine-learning model, and/or instantiating a neural network. Training data that may be used to train the machine-learning model and/or the neural network may include exemplary input data, such as without limitation, sensor signal input, sensor signal output, manipulated signal input, processed signal output, and the like, where each such example may be correlated to additional exemplary output data such as, without limitation, sensor signal output. Training of the model/network may take place either at the high-performance prosthetic attachment 100 or remotely; in the latter case, the model/network may be deployed at or by high-performance prosthetic attachment 100 in any manner as described within this disclosure. Additionally, in some embodiments, the machine-learning model and/or the neural network may be updated to high-performance prosthetic attachment 100, the model/network may be deployed at or by high-performance prosthetic attachment 100 in any manner as described in this disclosure. The machine-learning model and/or neural network may be deployed/instantiated once trained in any form as described within this disclosure. Feedback from the deployment of the machine-learning model and/or neural network may be turned into new training data, which may be stored either locally and/or transmitted to another device and used for retraining of the model/network. Retraining may be administered either remotely or at high-performance prosthetic attachment 100. Following the retraining of the model/network, redeployment/instantiation may be accomplished at or by high-performance prosthetic attachment 100 in any manner as described within this disclosure.

Continuing to reference FIG. 1A, control circuit 140 may be communicatively connected to high-performance prosthetic attachment 100 in any way disclosed within this disclosure. For example, and without limitation, control circuit 140 may be communicatively connected with at least a sensor 136 and/or other elements of high-performance prosthetic attachment 100. Additionally, control circuit 140 may be configured to receive attachment data and drive actuator 132 to adjust a range of movement available to at least an articulating attachment 104. Attachment data may be received from any embodiment of sensor 136 as described in this disclosure. In some embodiments, control circuit 140 may control range of motion of at least an articulating attachment 104 by way of mechanical positioning. Meaning, the placement of base connector 112, base 116, and/or plate, may restrict the range of motion in a way that is appropriate for a specific attachment.

With continued reference to FIG. 1A, in some embodiments control circuit 140 may instantiate a machine-learning model. A machine-learning model may be configured to receive at least an attachment datum and output a range of motion control for actuator and then drive actuator 132 using range of motion control. Additionally, machine-learning model may be configured to receive data from any sensor 136 as described in this disclosure. Instantiating a machine-learning model may function to improve efficiency and capability of high-performance prosthetic attachment 100. Attachment data and/or data from one or more sensors 136, may be received from any one or multiple sensors 136 as described in this disclosure.

Still referring to FIG. 1A, in some embodiments, control circuit 140 may instantiate a neural network. A neural network may be configured to receive at least an attachment datum and output a range of motion control for actuator and then drive actuator 132 using range of motion control. Additionally, a neural network may be configured to receive data from any sensor 136 as described in this disclosure. In some embodiments, instantiating a neural network may function to require less formal statistical training. Attachment data and/or data from one or more sensors 136, may be received from any one or multiple sensors 136 as described in this disclosure.

With continued reference to FIG. 1A, in some embodiments control circuit 140 may be configured to control at least an actuator 132 using a closed-loop control system, wherein the control system operates on iterative feedback from at least a sensor 136. In some embodiments a closed-loop control system may function as a reliable and accurate feedback system of input data. Attachment data and/or data from one or more sensors 136, may be received from any one or multiple sensors 136 as described in this disclosure.

With continued reference to FIG. 1A, an exemplary embodiment of control circuit 140 may include a computing device. Computing device may include a processor communicatively connected to a memory. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1A, computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a non-limiting example, using a “shared nothing” architecture.

With continued reference to FIG. 1A, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With further reference to FIG. 1A, control circuit 140 may be configured to receive attachment data and drive actuator 132 to adjust the range of motion available to articulating attachment 104. Attachment data may be sensed by any sensor 136 embodiment as described within this disclosure. Determination and application of specific ranges of movement to articulating attachment 104 may be improved and/or fine-tuned by using aggregations, instantiating a machine-learning model, and/or instantiating a neural network. Training data that may be used to train a machine-learning model and/or a neural network may include exemplary input data, such as without limitation, base 116 sensor 136 input, base connector 112 sensor 136 input, sensed attachment data, acceptable ranges of movement, actuator commands, and the like, where each such example may be correlated to additional exemplary output data such as, without limitation, sensor signal output commands, specific ranges of movement, and the like. Training of the model/network may take place either at the high-performance prosthetic attachment 100 or remotely; in the latter case, the model/network may be deployed at or by high-performance prosthetic attachment 100 in any manner as described within this disclosure. Additionally, in some embodiments, a machine-learning model and/or a neural network may be updated to high-performance prosthetic attachment 100, a model/network may be deployed at or by high-performance prosthetic attachment 100 in any manner as described in this disclosure. A machine-learning model and/or neural network may be deployed/instantiated once trained in any form as described within this disclosure. Feedback from the deployment of a machine-learning model and/or neural network may be turned into new training data, which may be stored either locally and/or transmitted to another device and used for retraining of the model/network. Retraining may be administered either remotely or at high-performance prosthetic attachment 100. Following retraining of a model/network, redeployment/instantiation may be accomplished at or by high-performance prosthetic attachment 100 in any manner as described within this disclosure.

Continuing to reference to FIG. 1A, control circuit 140 may be configured to receive sensor data relating to postural sway and/or postural balance and drive the actuator 132 to adjust a range of motion available to articulating attachment 104. Postural data may be sensed by any sensor 136 embodiment as described within this disclosure. Determination of adjustments of articulating attachment 104 in relation to postural data may be improved and/or fine-tuned by using aggregations, instantiating a machine-learning model, and/or instantiating a neural network. Training data that may be used to train the machine-learning model and/or the neural network may include exemplary input data, such as without limitation, base 116 sensor 136 input, base connector 112 sensor 136 input, sensed attachment data, acceptable ranges of movement, actuator commands, and the like, where each such example may be correlated to additional exemplary output data such as, without limitation, sensor signal output commands, specific ranges of movement, and the like. Additional exemplary input data may include, postural sway data, postural balance data, motion and gravity data such as without limitation, Euler angles, velocity, direction, amplitude, and the like, where each such example may be correlated to any previously listed exemplary output data as well as, without limitation, actuator adjustment commands and the like. Training of a model/network may take place either at high-performance prosthetic attachment 100 or remotely; in the latter case, a model/network may be deployed at or by high-performance prosthetic attachment 100 in any manner as described within this disclosure. Additionally, in some embodiments, a machine-learning model and/or a neural network may be updated to high-performance prosthetic attachment 100, a model/network may be deployed at or by high-performance prosthetic attachment 100 in any manner as described in this disclosure. A machine-learning model and/or neural network may be deployed/instantiated once trained in any form as described within this disclosure. Feedback from the deployment of a machine-learning model and/or neural network may be turned into new training data, which may be stored either locally and/or transmitted to another device and used for retraining of a model/network. Retraining may be administered either remotely or at high-performance prosthetic attachment 100. Following the retraining of the model/network, redeployment/instantiation may be accomplished at or by high-performance prosthetic attachment 100 in any manner as described within this disclosure.

Referring now to FIG. 1B, a top-down view of an exemplary embodiment of a high-performance prosthetic attachment 100 is shown. Within this top-down view at least an articulating attachment 104, limb connector 108, base connector 112, adjustment wheel 128, and control circuit 140 are illustrated.

Referring now to FIG. 1C, an exemplary embodiment of high-performance prosthetic attachment 100 is shown with an inline-skate-type base 116. In an embodiment, high-performance prosthetic attachment 100 may include one or more bases 116. In this particular embodiment, base 116 may be likened to an inline skate, otherwise known as a rollerblade. This embodiment's individual parts and attachments may be configured in any way as described within this disclosure.

Referring now to FIG. 1D, an exemplary embodiment of high-performance prosthetic attachment 100 is shown with an ice-skate-type base 116. In an embodiment, high-performance prosthetic attachment 100 may include one or more bases 116. In this particular embodiment, base 116 may be likened to an ice-skate. Certain cutouts of material of base 116 may be desired to configure a light-weight prosthetic in similar weight to a non-prosthetic user's skate. This embodiment's individual parts and attachments may be configured in any way as described within this disclosure.

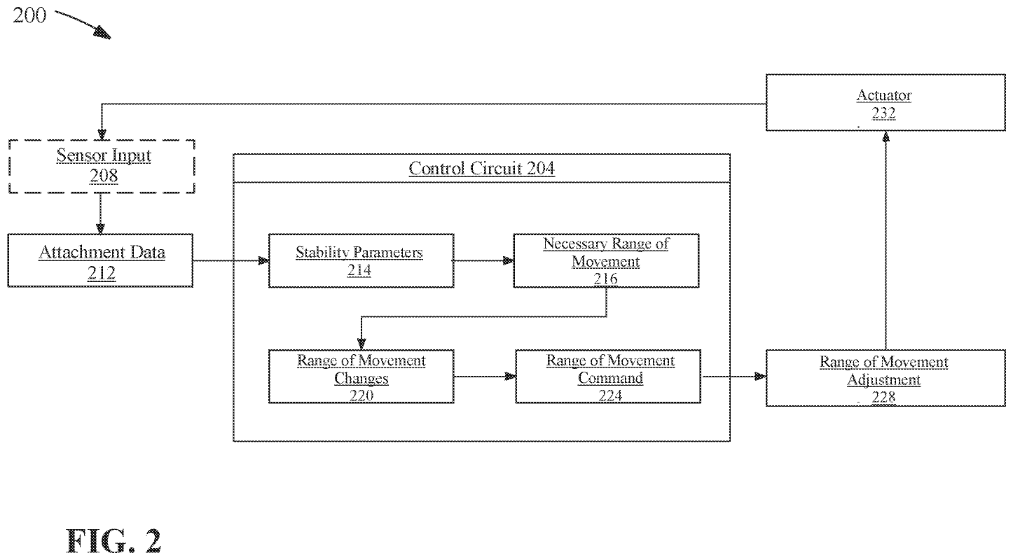

Referring now to FIG. 2, a block diagram showing an exemplary embodiment of a high-performance prosthetic attachment range of movement adjustment process 200. Sensor input 208 may come from any individual or combination of sensors as described within this disclosure. Sensed signals may be processed and relayed to control circuit 204, as attachment data 212. Attachment data 212 may be further processed by control circuit 204, taking into consideration stability parameters 214, necessary range of movement 216 for a given base, range of movement changes 220, and the ultimate range of movement command 224. This may be an output of control circuit 204 as a range of movement adjustment 228 to act on at least an actuator 232. Once the actuator has received a command and implemented it, the process may be reiterated continuously to facilitate participation in any given sport and provide associated balance and flexibility required for such participation.

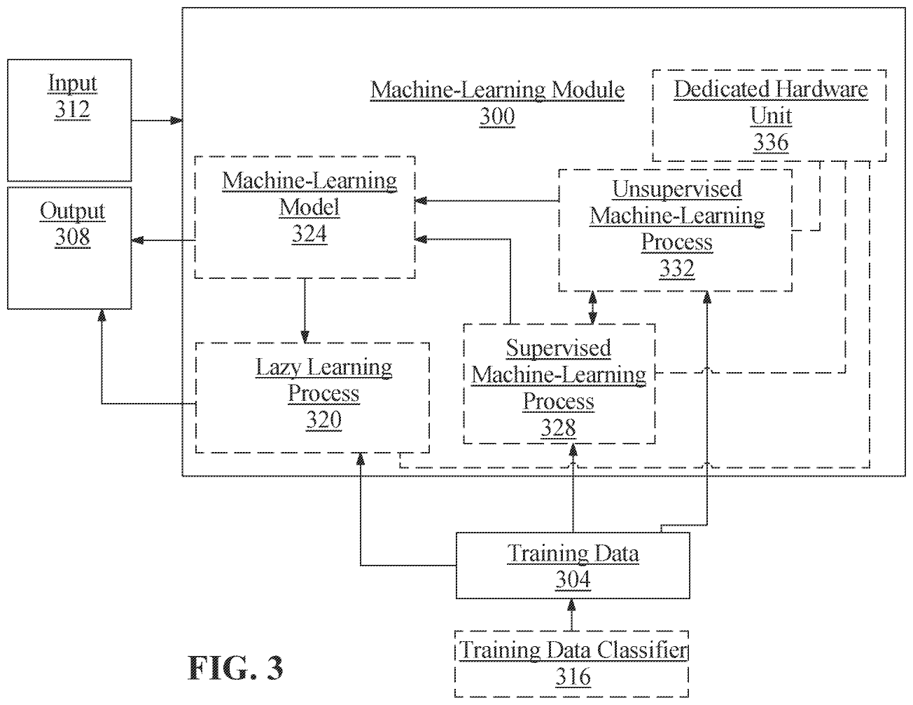

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example exemplary input data may include, postural sway data, postural balance data, motion and gravity data such as without limitation, Euler angles, velocity, direction, amplitude, and the like, where each such example may be correlated to any previously listed exemplary output data as well as, without limitation, actuator adjustment commands and the like.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification May be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to represent various base attachments and their correlated acceptable ranges of movement in order to drive the actuator to adjust the ranges of movement.

Still referring to FIG. 3, computing device 304 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P (A/B)=P (B/A) P (A)=P (B), where P (A/B) is the probability of hypothesis A given data B also known as posterior probability; P (B/A) is the probability of data B given that the hypothesis A was true; P (A) is the probability of hypothesis A being true regardless of data also known as prior probability of A; and P (B) is the probability of the data regardless of the hypothesis. A naïve Bayes algorithm may be generated by first transforming training data into a frequency table. Computing device 304 may then calculate a likelihood table by calculating probabilities of different data entries and classification labels. Computing device 304 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, computing device 304 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿ aᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively, or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively, or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 3, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include any input as described in this disclosure, any output as described in this disclosure, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

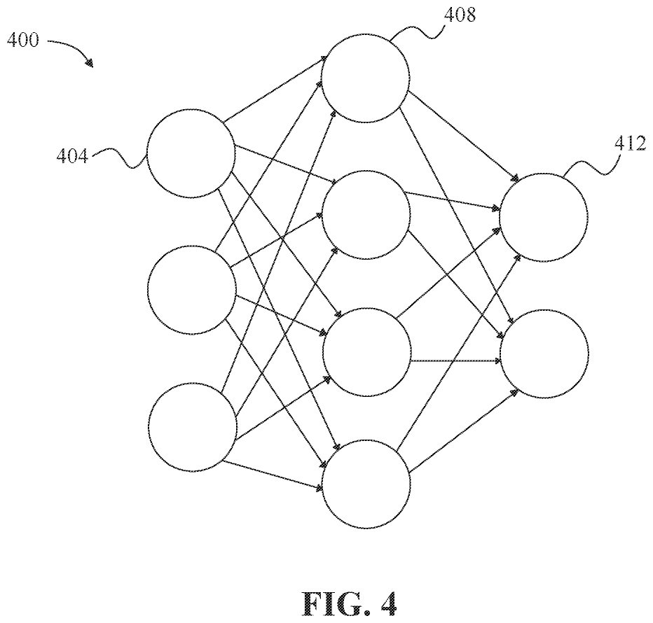

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

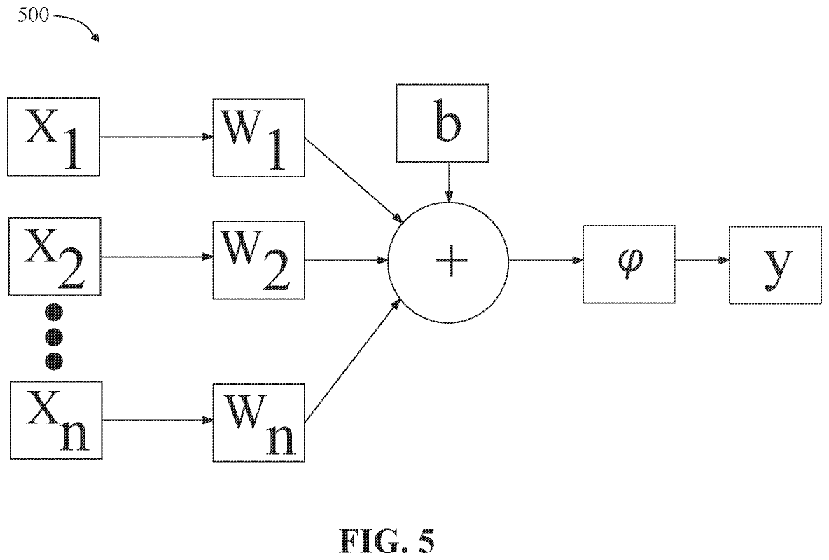

Referring now to FIG. 5, an exemplary embodiment of a node 500 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given
input x, a tanh (hyperbolic tangent) function, of the form

$$\frac{{e}^{x} - {e}^{- x}}{{e}^{x} + {e}^{- x}} ,$$

a tanh derivative function such as f(x)=tan h²(x), a rectified linear unit function such as f(x)=max(0, x), a “leaky” and/or “parametric” rectified linear unit function such as f(x)=max (ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid (x), a Gaussian error linear unit function such as f(x)=a(1+tan h(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

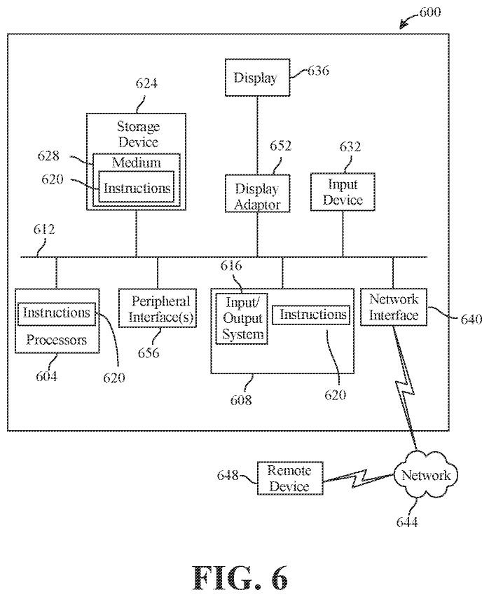

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A high-performance prosthetic attachment, the high-performance prosthetic attachment comprising:
at least one articulating attachment with varying, adjustable ranges of motion, comprising at least one adjustment wheel, wherein the adjustment wheel is located at a rear of the articulating attachment;
a limb connector coupled to the at least one articulating attachment, wherein the limb connector comprises a socket liner, wherein the socket liner comprises silicone elastomers, wherein the socket liner is tethered to an inside of a socket using a shuttle lock, wherein the limb connector comprises one or more forms of suspension, wherein a form of suspension within the one or more forms of suspension comprises a vacuum-assisted suspension;
a base connector configured to match a skate chassis coupled to the at least one articulating attachment, wherein the base connector comprises at least a sensor configured to scan at least a near-field communication tag identifying at least a characteristic of a base; and
the base, wherein the adjustment wheel is configured to adjust range of movement required to maintain stability and flexibility of the base.

2. The high-performance prosthetic attachment of claim 1, wherein the base connector is configured to be bolted into the base.

3. The high-performance prosthetic attachment of claim 1, wherein the base connector is configured to be bolted to a plate, and wherein the plate is further bolted to the base.

4. The high-performance prosthetic attachment of claim 3, wherein the plate comprises a metal material.

5. (canceled)

6. The high-performance prosthetic attachment of claim 3, wherein the plate comprises a carbon fiber material.

7. The high-performance prosthetic attachment of claim 1, wherein the at least one articulating attachment comprises a multi-axial joint.

8. The high-performance prosthetic attachment of claim 1, wherein the at least one articulating attachment comprises a hinged synovial joint.

9. The high-performance prosthetic attachment of claim 1, wherein the limb connector comprises a magnetic attachment.

10. The high-performance prosthetic attachment of claim 1, wherein the limb connector comprises at least a latching mechanism selected from a group consisting of a threaded adapter, an adhesive, and a bonding.

11. The high-performance prosthetic attachment of claim 1, wherein the base connector comprises a magnetic attachment.

12. The high-performance prosthetic attachment of claim 1, wherein the base connector comprises a carbon fiber material.

13. The high-performance prosthetic attachment of claim 1, further comprising:
at least one actuator mechanically connected to the at least an articulating attachment, wherein the at least one actuator is configured to adjust the range of motion of the at least an articulating attachment;
at least one sensor, wherein the at least one sensor is configured to detect an attachment datum of a base; and
a control circuit communicatively connected to the prosthetic attachment, wherein the control circuit is configured to:
receive the attachment datum; and
drive the at least one actuator to adjust the range of motion available to the at least one articulating attachment.

14. The high-performance prosthetic attachment of claim 13, wherein the at least one sensor is located on the base.

15. The high-performance prosthetic attachment of claim 13, wherein the at least one sensor is located on the at least one articulating attachment.

16. The high-performance prosthetic attachment of claim 13, wherein the at least one sensor is located on the base connector.

17. The high-performance prosthetic attachment of claim 13, wherein the at least a sensor comprises one sensor suite.

18. The high-performance prosthetic attachment of claim 13, wherein the at least one sensor is located both on the at least one articulating attachment and on the base connector.

19. The high-performance prosthetic attachment of claim 13, wherein the control circuit is further configured to instantiate a machine learning model.

20. The high-performance prosthetic attachment of claim 19, wherein the machine learning model comprises a neural network.
